(19) United States
(12) Patent Application Publication
(10) Pub. No.: US 2014/0239198 A1
(43) Pub. Date: Aug. 28, 2014

(54) EXTERNAL BEAM RADIATION THERAPY FOR A PLURALITY OF COMPARTMENTS

(71) Applicant: Moshe Ein-Gal, Ramat Hasharon (IL)

(72) Inventor: Moshe Ein-Gal, Ramat Hasharon (IL)

(21) Appl. No.: 13/775,375

(22) Filed: Feb. 25, 2013

(51) Int. Cl. A61N 5/10

(52) U.S. Cl. CPC A61N 5/1077
USPC 250/492.1

(57) ABSTRACT

An external beam radiation therapy system including a plurality of compartments separated from one another by radiation shields, each of the compartments including a patient support system that includes a support member and a fixation member for supporting and spatially fixing a target portion of the patient for irradiation thereof, and a radiation source housing including at least one radiation source operable to emit radiation beams into the compartments towards the target portion in each of the compartments, wherein each of the compartments includes a beam shaper for shaping the radiation beams emitted into that compartment, and wherein the radiation shields are configured to provide adequate shielding in each compartment, in accordance with a safety standard, against radiation produced in other compartments.

### FIELD OF THE INVENTION

The present invention relates generally to radiotherapy and irradiation systems, and particularly to a system and a method for external beam radiation therapy, in particular for treatments of several patients.

### BACKGROUND OF THE INVENTION

External beam radiation therapy (EBRT) involves irradiating a target in a patient with external beams, typically of megavoltage photons. Historically, such beams have been produced by cobalt teletherapy devices which use a radioactive source of Co⁶⁰ isotope. The radioactive source is shielded and is mounted on a gantry that rotates in a vertical plane and emits a beam oriented towards a horizontally positioned patient.

Linear accelerators (Linacs) have been preferred to cobalt irradiators due to their higher energy (which has the advantage of deeper penetration), higher intensity (which has the advantage of shorter treatment times) and lower penumbra (which has the advantage of higher quality of dose delivery). Linac radiation beams are produced by accelerating electrons toward a target in a vacuum. Linacs are typically more expensive than Co-60 irradiators and require more sophisticated maintenance.

“Turning off” a radiation beam, that is, making the system emit no radiation beam to the target, is done electronically in Linacs and mechanically in cobalt teletherapy devices, such as by moving the source away from the primary collimator into a shielded location. This can also be accomplished by using attenuating shutters to block the beam.

EBRT shielding is done for both inside and outside the treatment room or area. For inside the treatment room, a source housing (which basically surrounds the source except for where the beam is emitted) provides protection against undesired radiation. For outside the treatment room, a system housing (e.g., vault) provides protection. Adequate shielding is in accordance with international and local standards.

The source housing typically includes an attenuating container surrounding the source and a fixed primary collimator as the only passageway for the radiation beam. Additional collimators may be provided to shape the beam aperture.

The vault protects not only against the direct (primary) beam but also against secondary radiation produced by leakage from the irradiating device and its beam shapers, as well as radiation scattered from the treated patient, the room walls, etc.

A typical EBRT treatment is sequenced into multiple temporal fractions. Fraction irradiation time is in the range of several minutes. Increasing the delivered dose rate reduces fraction irradiation time and may increase the EBRT system throughput.

In arc radiotherapy a patient is continuously irradiated while the beam is rotated about the patient in a plane generally perpendicular to the patient. A complete fraction can be delivered by an arc subtending a full circle, which reduces treatment time. Although additional sequentially-delivered conical arcs (i.e., where the beam is not perpendicular to the patient) may improve dose delivery, such additional arcs are not usually implemented for body radiotherapy.

### SUMMARY OF THE INVENTION

The present invention seeks to provide a novel system and method for external beam radiation therapy, as is described hereinbelow.

There is thus provided in accordance with an embodiment of the present invention an external beam radiation therapy system including a plurality of compartments separated from one another by radiation shields, each of the compartments including a patient support system that includes a support member and a fixation member for supporting and spatially fixing a target portion of the patient for irradiation thereof, and a radiation source housing including at least one radiation source operable to emit radiation beams into the compartments towards the target portion in each of the compartments, wherein each of the compartments includes a beam shaper for shaping the radiation beams emitted into that compartment, and wherein the radiation shields are configured to provide adequate shielding in each compartment, in accordance with a safety standard, against radiation produced in other compartments.

In one non-limiting embodiment the radiation beams are generally horizontal, and wherein for at least one of the compartments, the patient support system is operable to rotate the patient about a generally vertical rotational axis.

In one non-limiting embodiment the at least one radiation source includes a linear accelerator operable to sequentially deflect an accelerated electron beam in vacuum toward radiation-producing targets, wherein each of the radiation-producing targets is directed towards a specific compartment of the compartments.

In one non-limiting embodiment the at least one radiation source is rotatable about a rotational axis and configured to emit radiation beams towards a specific compartment of the compartments during rotation of the at least one radiation source.

In one non-limiting embodiment the at least one radiation source emits radiation beams omnidirectionally so as to emit radiation beams simultaneously to each of the compartments.

In one non-limiting embodiment a radiation blocker is provided for selectively blocking at least one of the radiation beams from being emitted into at least one of the compartments.

In one non-limiting embodiment at least one of the support members is configured to support the patient in one of the compartments at a different orientation than another of the support members in another of the compartments.

In one non-limiting embodiment, the system is used in a method for EBRT to irradiate patients in different compartments. The patients may be irradiated generally simultaneously or sequentially.

### BRIEF DESCRIPTION OF THE DRAWINGS

The present invention will be understood and appreciated more fully from the following detailed description, taken in conjunction with the drawings in which:

FIG. 1 is a simplified schematic illustration of an external beam radiation therapy system, constructed and operative in accordance with an embodiment of the present invention; and

FIG. 2 is a simplified schematic illustration of one of the compartments in the EBRT system of FIG. 1, in accordance with an embodiment of the present invention.

### DETAILED DESCRIPTION OF EMBODIMENTS

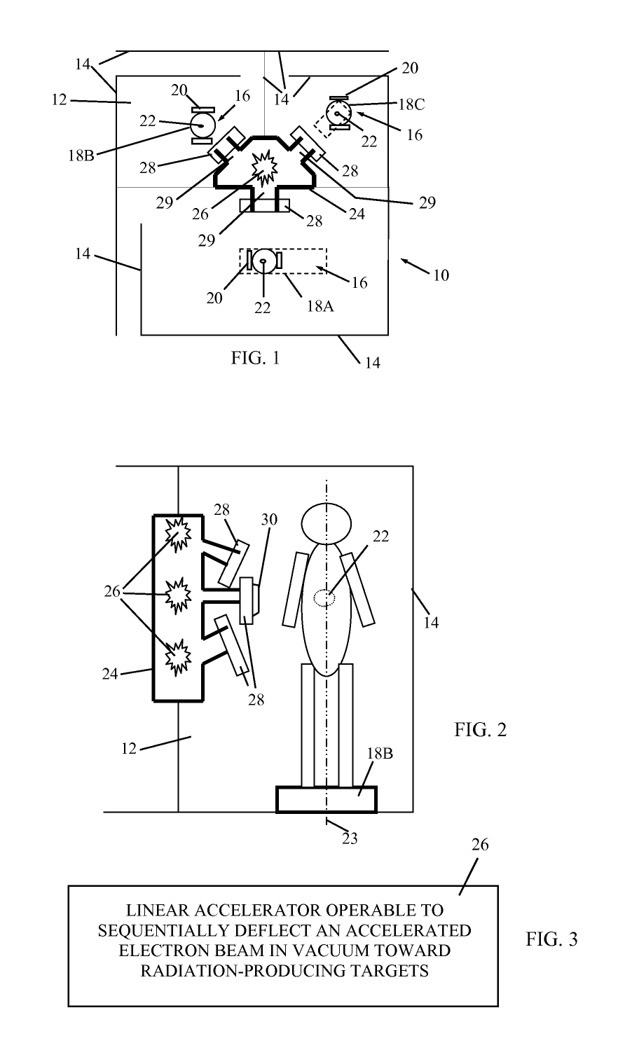

Reference is now made to FIG. 1, which illustrates an external beam radiation therapy system 10, constructed and operative in accordance with a non-limiting embodiment of the present invention.

EBRT system 10 includes a plurality of compartments 12 which are separated from one another by radiation shields 14. The radiation shields 14 are configured to provide adequate shielding in each compartment, in accordance with a safety standard, against radiation produced in other compartments. Each compartment is adequately shielded against secondary radiation produced in other compartments. In each compartment 12 there is a patient support system 16 that includes a support member and a fixation member 20 for supporting and spatially fixing a target portion 22 of the patient for irradiation thereof. The support member may be a horizontal, rotatable bed 18A upon which the patient lies, or a standing turntable 18B upon which the patient stands, or a sitting turntable 18C upon which the patient sits. In all of these examples, the patient may be rotated about a generally vertical rotational axis 23 (FIG. 2). The rotational axis 23 generally intersects the radiation beams associated with the compartment.

As seen in FIG. 1, each compartment 12 of EBRT system 10 may include a different support member (18A-18C) for supporting the patient at different orientations (e.g., lying, standing, sitting, or reclining) than another compartment. This adds to the versatility of the system and broadens its ability to handle different medical applications.

The fixation members 20 may be standard fixation components of stereotactic radiosurgery systems, such as clamps, nails, screws, etc.

EBRT system 10 includes a radiation source housing 24, which has one or more radiation sources 26 (e.g., Co⁶⁰ isotope) which can emit radiation beams into compartments 12 towards the target portion 22 in each of compartments 12. Any radiation may be used, such as but not limited to, heavy particles radiation or photon radiation (gamma radiation).

Each compartment 12 includes one or more beam shapers 28 (e.g., a cylindrical collimator, multileaf collimator, physical compensator or others) for shaping the radiation beams emitted into that compartment. All collimators in a compartment are operable to direct respectively corresponding radiation beams to a common location in the compartment, a location in which the target to be treated is positioned. Patient support and collimators in a compartment may be tailored to a specific application where the patient may be standing, seating, leaning or lying down. The resolution and field size of the collimators may also be optimized for the application, as well as the position of the rotational axis 23 relative to source housing 24. Optimizing respective compartments for specific applications may allow simultaneous treatments, such as prostate, lung and breast.

In one embodiment, the radiation source 26 is a radioactive source (e.g., cobalt-60 source), which emits radiation in a multiplicity of orientations (e.g., omnidirectionally). In another embodiment, the radiation source 26 may be a linear accelerator that sequentially emits radiation beams in desired orientations. For example, the radiation beams may be produced by an electron gun, accelerated by the accelerating structure with microwave pulses from a magnetron and then electron optics deflect accelerated electrons in vacuum toward radiation-producing targets. The impinging electrons on each target produce respective radiation beams that are directed to the different compartments. Fast sequencing of these beams is practically equivalent to simultaneous irradiation from an omnidirectional radiation source. In another embodiment, the radiation source housing 24 is rotated so that the radiation beams are sequentially directed to the different compartments.

In another embodiment, one or more radiation blockers 30 are provided for selectively blocking the radiation beam from being emitted into the compartment. Blocker 30 may be a shutter that selectively blocks the beam. Alternatively, the radiation beams may be blocked by moving the radiation source away from the beam shaper 28 as is typically done with prior art teletherapy cobalt-60 devices.

Imaging apparatus (not shown), such as a fluoroscope or ultrasound apparatus, for example, may be provided for imaging the target irradiated by the radiation beams.

The scope of the present invention includes both combinations and subcombinations of the features described hereinabove as well as modifications and variations thereof which would occur to a person of skill in the art upon reading the foregoing description and which are not in the prior art.

1. An external beam radiation therapy (EBRT) system comprising:
a plurality of compartments separated from one another by radiation shields, each of said compartments comprising a patient support system that comprises a support member and a fixation member for supporting and spatially fixing a target portion of the patient for irradiation thereof; and
a radiation source housing comprising at least one radiation source operable to emit radiation beams into said compartments towards the target portion in each of said compartments, wherein each of said compartments comprises a beam shaper for shaping the radiation beams emitted into that compartment, and wherein said radiation shields are configured to provide adequate shielding in each compartment, in accordance with a safety standard, against radiation produced in other compartments, and wherein said at least one radiation source emits radiation beams omnidirectionally through different passages towards each of said compartments.

2. The system according to claim 1, wherein said radiation beams are generally horizontal, and wherein for at least one of said compartments, said patient support system is operable to rotate the patient about a generally vertical rotational axis.

3. The system according to claim 1, wherein said at least one radiation source comprises a linear accelerator operable to sequentially deflect an accelerated electron beam in vacuum toward radiation-producing targets, wherein each of said radiation-producing targets is constructed so as to allow radiation to be directed towards a specific compartment of said compartments.

4. The system according to claim 1, wherein said at least one radiation source is rotatable about a rotational axis and configured to emit radiation beams towards a specific compartment of said compartments during rotation of said at least one radiation source.

5. The system according to claim 1, wherein said at least one radiation source emits radiation beams in a multiplicity of orientations so as to emit radiation beams simultaneously to each of said compartments.

6. The system according to claim 1, further comprising a radiation blocker for selectively blocking at least one of said radiation beams from being emitted into at least one of said compartments.

7. The system according to claim 1, wherein at least one of said support members is configured to support the patient in one of said compartments at a different orientation than another of said support members in another of said compartments.

8. A method for EBRT comprising using the system of claim 1 to irradiate patients in different compartments.

9. The method according to claim 8, wherein the patients are irradiated generally simultaneously.

10. The method according to claim 8, wherein the patients are irradiated generally sequentially.
